(19) United States
(12) Patent Application Publication
(10) Pub. No.: US 2008/0102139 A1
(43) Pub. Date: May 1, 2008

(54) Pharmaceutical Formulation for Treatment of Acquired Immune Deficiency Syndrome (AIDS).

(76) Inventor: Jose Ramon Baez Acosta, Santo Domingo (DO)

Correspondence Address: FURR LAW FIRM, 2622 DEBOLT ROAD, UTICA, OH, 43080, US

(21) Appl. No.: 11/843,154

(22) Filed: Aug. 22, 2007

(30) Foreign Application Priority Data
Oct. 26, 2006 (DO) P2006-0236

(51) Int. Cl. A61K 36/88; A61P 31/18

(52) U.S. Cl. 424/725

(57) ABSTRACT

Present invention is related to pharmaceutical branch and in particular to an extract obtained from a purple maguey (Tradescantia Spathacea) plant be used in the elaboration of pharmaceutical formulations applied to immune deficiency syndrome (AIDS) treatment. Extract obtaining procedure is also illustrated.

### INVENTION BACKGROUND

1. Technical Field of the Invention

This invention is involved with Pharmaceutical branch in general and in particular with a substance obtained out of purple maguey (Tradescantia Spathacea) that allows getting pharmaceutical formulations useful to treat Acquired Immune Deficiency Syndrome (AIDS), as well as its obtaining process.

2. Description of Previous Technique State

It is widely known that this pandemic is still growing and what is a serious problem: there are some countries which exhibited sustainable low rates of AIDS infection or even rates of infection tending to decrease, and these countries have experienced an increase in recent times.

According to a recent report issued by AIDS UNO/WHO about the situation of AIDS pandemic in 2006, it is estimated that more than 40 millions of people are living with AIDS in the world; 4.3 millions of new infections occurred just in 2006, 65% of them (2.8 millions) were located in Sub-Saharan Africa what comes to be added to the dramatic increases in countries of Eastern Europe and Central Asia where there are some clues indicating an increase in infection rates in more that 50% just from 2004. Just in 2006 2.9 millions of people died due to several diseases related to AIDS.

Up to this moment AIDS has no cure and about 15 medicines are being used to treat the infection. Treatment includes the combination of several antiretroviral medicines that avoid immunological depression and stop virus multiplication. Antiretroviral therapy is complex and expensive since it includes the administration of at least three medicines (triple therapy) several times a day and high doses that produces non desired effects interacting with other medicines that should be taken with or without meals. But what is worst is the fact that this therapy does not guarantee the total cure and the elimination of the disease.

Beside antiretroviral therapy mentioned above, there is a study about therapeutic properties of some plants that could be really used for getting a final cure for this terrible disease, and it is precisely in this technological field in which this invention is framed in.

On the other hand, curative properties of maguey in general are well known and in particular its applications to gastrointestinal and skin diseases also, what has generated a great interest in the study of other therapeutic applications of these plants lately.

Patent MXJL04000017 is known in technique state and today it is in application stage under the title of “MAGUEY (AGAVE SALMIANA) PLANT EXTRACT FOR CURING HUMAN VIRUS DISEASES” with the priority number MX2004JL00017 20040707. The applicant's name is BELTRAN JOSE GARIBAY. Use of maguey extract to cure viral diseases like AIDS is restored in this study. A method to obtain the extract is included in this patent which consists in pressing the plant (previously washed) with metal rolls to extract its juice which is kept and ready to be used. However, it is known all maguey species are commonly toxic, so its application in human beings is not possible in a direct way without generating a toxic reaction pretty aggressive.

Tradescantia Spathacea plant or purple maguey belongs to the Commelinaceae family composed by 44 genres and about 600 species, is a herb with thick silky juicy dark purple (on the opposite side) leaves, its height is about 20 and 25 cm and its width about 3.5 cm. Its stem is about 20 cm height, white three petals (5 to 8 mm) flowers and rough seeds (3 mm length and 1.5 mm width). This plant has gone through several trials since early 90s and it has been possible to state that this plant contains some substances known as flavonics and cumarics which have anti-inflammatory properties. Recent researches focus on the plant have revealed that it also eliminates formation of tumors, and have been successfully tested on mama tissue, prostate, skin, colon and tumor affections related to leukemia.

Already mentioned uses related to Tradescantia Spathacea are listed in the technique state as well as its use in the elaboration of cosmetics and pharmaceutical products focused on dermatologic appliances as it is stated in patent WO9909945 entitled “USE OF THE RHOEO DISCOLOR PLANT EXTRACTS IN COSMETICS AND PHARMACEUTICS, IN PARTICULAR IN DERMATOLOGY” whose applicant is CENTRE NAT RECH SCIENT from France.

Besides the properties and applications mentioned above, researcher has discovered Tradescantia Spathacea is particularly useful in treatments related to AIDS, and it is not previously known or reported this application in the technique state.

### DESCRIPTION OF THE INVENTION

Present invention main object is to provide a procedure to obtain a substance out of purple maguey (Tradescantia Spathacea).

A second object is to provide pharmaceutical formulations from purple maguey extract (Tradescantia Spathacea), used to eliminate acquired immune deficiency syndrome AIDS.

A third object deals with a treatment based on the pharmaceutical formulation got out of present invention in the treatment of AIDS patients.

Getting purple maguey extract (Tradescantia Spathacea) begins with the use of the whole plant which goes through a washing process using purified water to eliminate impurities, then the plant is cut into pieces of 3 or 4 inches to be introduce in a professional liquidizer to get the plant juice. After this, extract is filtered to eliminate fibers and final juice is boiled in a stainless recipient to eliminate existing water in order to obtain a pasty black yellowish substance.

Researcher has discovered this final product obtained out of purple maguey (Tradescantia Spathacea) is useful to fight against infections caused by human immune deficiency virus. Tests done to animals in lab allowed to check animals infected with AIDS which received a treatment with purple maguey extract injections and several doses of the extract after a period of time were totally healthy, what was supported by lab tests and the application of BIO-Card HIV1/2/0 (Fast Test for Qualitative Detection of Antibodies HIV) and even by ELISA methods.

However, extract of purple maguey has a pasty and dense consistency which makes no possible its application directly on a living being. Also it is considered to be toxic and irritant. So, it has been mixed with hydrochlorate of lidocaine because it is necessary to reduce toxicity and to avoid non desired reactions in patients. Then, to improve moisturizing properties it should be mixed with propelinglicol and to be applied in injections should be mixed with water injection. In this way every mixture becomes a different pharmaceutical formulation from the same extract.

An example of the action spectrum of the present invention is given.

To get extract from purple maguey (Tradescantia Spathacea) it is necessary to select the whole plant, wash it during 10 minutes using purified water till impurities are totally removed. Then the plant is cut into pieces of 3 inches and it is put into an industrial mixer to triturate it in order to get an extract with fibers which are eliminated once this extract if filtered. After filtering process, obtained extract is kept in a stainless recipient and is boiled to evaporate as much water as possible. The final result is a black yellowish paste.

A pharmaceutical formula (injection 1 mL) that can be obtained from purple maguey extract (Tradescantia Spathacea) is given with an illustrative and no limitative character. This formula has the following composition:

| | | |
|---|---|---|
| Tradescantia Spathacea extract | 250 | mg. |
| USP Propilenglicol. | 3 | mg. |
| hydrochlorate of lidocaine | 1 | mg. |
| Injection water c.s.p. | 1 | ml. |

Other pharmaceutical formulations can be gotten from mentioned extract, so it is not limited to the injection formula given above. New formulations can be powders, capsule, tablets, coated pills, creams, gels, solutions, injections, ointment and syrups.

1. Pharmaceutical formulation for treatment of Acquired Immune Deficiency Syndrome (AIDS). Obtaining procedure characterized by getting it out of purple maguey extract (Tradescantia Spathacea).

2. Pharmaceutical formulation for treatment of Acquired Immune Deficiency Syndrome (AIDS). Obtaining procedure according to claim 1 characterized by its composition since active ingredient is purple maguey extract (Tradescantia Spathacea), USP Propilenglicol, Hydrochlorate of lidocaine, and injection water.

3. Pharmaceutical formulation for treatment of Acquired Immune Deficiency Syndrome (AIDS). Obtaining procedure according to claims 1 and 2 characterized by 1 mL intramuscular vaccine containing proportions of 250 mg of Tradescantia Spathacea extract, 30 mg USP Propilenglicol, 1 mg Hydrochlorate of lidocaine and 1 mL of injection water.

4. Pharmaceutical formulation for treatment of Acquired Immune Deficiency Syndrome (AIDS). Obtaining procedure characterized by getting the extract out of a selected whole plant which is carefully washed using purified water. then plant is cut into pieces, liquidized, filtered and boiled till getting a black yellowish pasty substance.

5. Pharmaceutical formulation for treatment of Acquired Immune Deficiency Syndrome (AIDS). Obtaining procedure according to claims 1, 2, and 3, is characterized by the application of a quantity therapeutically appropriate of purple maguey (Tradescantia Spathacea) extract to an AIDS virus infected patient.
